(19) United States
(12) Patent Application Publication
(10) Pub. No.: US 2024/0003887 A1
(43) Pub. Date: Jan. 4, 2024

(54) FREE PROSTATE SPECIFIC ANTIGEN MEASUREMENT KIT AND PREPARATION METHOD THEREFOR

(71) Applicant: Beijing Strong Biotechnologies, Inc., Beijing (CN)

(72) Inventors: Xiaorui Zhang, Beijing (CN); Jingjing Zhang, Beijing (CN); Huaya Cai, Beijing (CN); Xi Liu, Beijing (CN)

(21) Appl. No.: 17/766,943

(22) PCT Filed: Oct. 30, 2020
(86) PCT No.: PCT/CN2020/125263

§ 371 Date: Apr. 6, 2022

(30) Foreign Application Priority Data
Nov. 6, 2019 (CN) 201911077239.8

(51) Int. Cl. G01N 33/574; G01N 33/577; G01N 33/531; G01N 33/543

(52) U.S. Cl. CPC G01N 33/57434; G01N 33/577; G01N 33/531; G01N 33/54346; G01N 2333/96433

(57) ABSTRACT

A free prostate specific antigen measurement kit and a preparation method therefor. The kit comprises a first reagent and a second reagent, and optionally further comprises a quality control material and/or a calibration material. The first reagent comprises a surfactant and a buffer solution, and the second reagent comprises nanoparticles coated with an antibody and a buffer solution. The kit uses a free prostate specific antigen in a sample to react with an antibody in the second reagent.

### FIELD OF THE INVENTION

The present application belongs to the field of in vitro clinical diagnosis and medical immunology, and relates to an immune detection reagent. Further, the present application relates to a detection kit for fPSA.

### BACKGROUND OF THE INVENTION

Human prostate-specific antigen (hereinafter referred to as PSA) is a single-chain glycoprotein secreted by epithelial cells of prostate acinus and duct, with a molecular weight of about 34KD. PSA is functionally a serine protease (similar to kallikrein) and is involved in the liquefaction process of semen. It is an essential indicator routinely used in clinic for the diagnosis and identification of benign and malignant prostate diseases, and for postoperative follow-up of patients with prostate cancer.

Under normal physiological conditions, PSA is secreted into the semen through the duct. The concentration of PSA in semen is one million times higher than that in serum. There is a obvious tissue barrier between the prostate acinus and duct lumen, and the blood circulatory system. The tissue barrier will be damaged to varying degrees when suffering from prostate disease. Especially when suffering from prostate cancer, such natural barrier will be seriously damaged due to the abnormal growth of tumor cells, resulting in the leaking of a large amount of PSA into the blood, and a substantial increase in serum PSA level.

Studies have shown that PSA exists in two forms in the blood circulation: conjugated PSA (cPSA) accounting for more than 85%, and free PSA (i.e. fPSA) accounting for about 15%, and the sum of the two refers to total PSA (tPSA).

In clinical practice, tPSA>4 ng/ml is usually used as the threshold value for screening prostate cancer; the tPSA result between 4 and 10 ng/ml is deemed as the gray area, and both prostate cancer and benign prostatic hyperplasia are possible; and when tPSA>10 ng/ml, prostate cancer is highly possible.

Inconsistency is reported in literatures with respect to the ratio of fPSA/tPSA. Some reports a threshold value of 0.16, and others report a threshold value of 0.19 or 0.25. fPSA/tPSA is very important when serum tPSA falls in the gray area. Where fPSA/tPSA is greater than the threshold value, it is less possible to be prostate cancer; whereas when the fPSA/tPSA value is less than the threshold value, it is highly possible to be diagnosed as prostate cancer.

The demand for laboratory tests has increased, since medical professionals and patients realize the potential value of fPSA in the diagnosis of prostate cancer, leading to a requirement for faster, more accurate, and more effective detection methods provided in the field to help doctors and patients get test results earlier.

Currently, fPSA is usually determined by immunological methods. Commonly used detection methods include:
(1) Chemiluminescence immune quantitative assay, which is a technology for quantitative detection via direct luminescence by an automatic control system, using magnetic microparticle-conjugated PSA solid-phase reagent and mAb PSA liquid-phase reagent, and acridinium ester as labeling reagent. The technology has high sensitivity and strong specificity, but the instruments and reagents are expensive and not suitable for screening at primary level;
(2) Enzyme labeling assay (EIA), which is usually for the measurement of f-PSA, c-PSA and t-PSA by double-antibody sandwich method. Antibodies used are monoclonal antibodies against different epitopes on PSA; different types of PSA can be detected with the corresponding monoclonal antibodies. The disadvantage of this method is the poor repeatability, and the high requirement for the operators;
(3) Radioimmunoassay (RIA), which has environmental pollution issues;
(4) Gold-labeling analysis, which has advantages such as being easy to use, easy to read out, rapid response, while the accuracy is low.

The problem to be solved in present application is to overcome the defects existing in the above-mentioned existing reagents, and to provide a new detection kit for the measurement of the content of fPSA in a sample (e.g. serum or plasma) in latex-enhanced turbidimetric immunoassay, thereby improving the detection speed, reducing the operational complexity, and reporting reliable results as soon as possible.

### SUMMARY OF THE INVENTION

According to an aspect of the present application, there is provided a detection reagent comprising a first antibody and a second antibody; the first antibody is an anti-antigen antibody; the second antibody is an anti-complex antibody; wherein the second antibody does not bind the antigen in free status, but binds to the complex which is formed by the first antibody and said antigen.

In some embodiments, the antigen is human fPSA.

In some embodiments, the first antibody is a monoclonal antibody or antigen-binding fragment thereof; the second antibody is an antigen-binding fragment. Monoclonal antibodies are derived from: murine, leporidae, avian, caprinae, recombinant antibody. The antigen-binding fragment is selected from the group consisting of: Fab, Fab′, F(ab′)2, scFv, Fv, dsFv and single domain antibody;

According to another aspect of the present application, there is provided a fPSA detection kit, comprising a first reagent and a second reagent.

In some embodiments, the first reagent comprises a surfactant and a buffer.

In some embodiments, the second agent comprises:
a first nano-microsphere coated with a first antibody,
a second nano-microsphere coated with a second antibody, and
a buffer.

In some embodiments, the buffers comprised in the first and second reagents are each independently selected from the group consisting of: phosphate buffer, glycine buffer, HEPES buffer, MES buffer (also called 2-morpholine ethanesulfonic acid buffer), boric acid buffer, acetate buffer and ammonium chloride buffer.

In some embodiments, the concentration of the buffer is 10-500 mM, preferably, the concentration is 20 mM, 30 mM, 40 mM, 50 mM, 60 mM, 70 mM, 80 mM, 90 mM, 100 mM, and a range between any two of the above values; as an example, the concentration of the buffer is 20 mM, 30 mM, 40 mM, 50 mM, and a range between any two of the above values.

In some embodiments, the pH value of the buffer is 6 to 8, as an example, pH is 7, 7.1, 7.2, 7.3, 7.4, 7.5 and a range between any two of the above values.

In some embodiments, the types of the buffers comprised in the first and second reagents may be the same or different.

In some embodiments, the concentrations of the buffers comprised in the first and second reagents may be the same or different.

In some preferred embodiments, the buffer type of the first reagent is the same as that of the second reagent; and the buffer concentration of the first reagent is different from that of the second reagent.

In a specific embodiment, the buffer of the first reagent is 50 mM HEPES buffer; in a specific embodiment, the buffer of the second reagent is 20 mM glycine buffer. Those skilled in the art can understand that the pH values can vary depending on factors such as buffer type, concentration.

In some embodiments, the first antibody is a monoclonal antibody or antigen-binding fragment thereof; the second antibody is an antigen-binding fragment. Monoclonal antibodies are derived from: murine, leporidae, avian, caprinae, recombinant antibody. The antigen-binding fragment is selected from the group consisting of: Fab, Fab′, F(ab′)2, scFv, Fv, dsFv and single domain antibody;

In a specific embodiment, it is not recommended to use a complete monoclonal antibody as the second antibody, due to its relatively large molecular weight. In specific embodiments, the second antibody is an antigen-binding fragment having a smaller molecular weight than a monoclonal antibody, such as a single domain antibody (an antibody derived from camelid, such as alpaca).

In some specific embodiments, the second antibody is linked to the second nano-microsphere via a spacer arm molecule. The spacer arm molecule is glutaraldehyde or an inert carrier protein. The inert carrier protein is selected from the group consisting of: serum albumin, thyroglobulin, ceruloplasmin, ovalbumin and polylysine.

In some specific embodiments, when the first antibody is a monoclonal antibody, the second antibody is a single domain antibody.

In other specific embodiments, when the first antibody is an antigen-binding fragment, the second antibody is an antigen-binding fragment (selected from the group consisting of: Fab, Fab′, F(ab′)2, scFv, Fv, dsFv and single domain antibody).

In some embodiments, the first agent comprises one or more selected from the following:
0.1 to 0.5M NaCl,
0.05 to 0.2% (w/v) preservative,
0.05 to 0.2% (w/v) BSA,
0.5 to 2% (w/v) PEG2000-PEG80000 (e.g. 6000), and
0.1% to 0.5% (w/v) AEO7.

It should be understood that although the specific concentrations of the various components in the reagents are disclosed in present application, skilled persons have an ability to prepare the reagents in various concentrated or diluted forms, and thus the concentrated and diluted forms of the reagents still fall within the scope of present application.

As required, the fPSA detection kit according to the present application further comprises a quality control and/or a calibrator. The calibrator is primarily used for calibrating the measurement system, evaluating measurement procedures, or assigning values to samples to be tested. Thus, the calibrator comprises fPSA with known concentration, and the value of the calibrator can even be traced back to a reference substance or to a reference method (NIBSC 96/668). Based on the concentration range of the substance to be tested, those skilled in the art can prepare calibrators with appropriate concentrations by using methods commonly used in the art, or can use commercially available calibrators (for example, fPSA purity national standard material No. 150544-200702), or can use working calibrator provided by manufacturers.

In some specific embodiments, the fPSA detection kit of the present application also comprises several different concentrations of calibrators, such as but not limited to 2, 3, 4, 5, or even more calibrators of different concentrations.

In one embodiment, the fPSA detection kit of the present application comprises calibrators with 6 different concentrations. The calibrator comprises fPSA (such as but not limited to 0 ng/ml, 0.5 ng/ml, 1 ng/ml, 2 ng/ml, 5 ng/ml and 10 ng/ml), buffer, when appropriate stabilizer (such as BSA), or preservative (such as NaN₃), etc.

The calibrator can be prepared in the form of liquid, dry powder or lyophilized powder.

The buffer comprised in the calibrator is selected from the group consisting of: phosphate buffer, glycine buffer, HEPES buffer, MES buffer, boric acid buffer, acetate buffer and ammonium chloride buffer; the concentration of the buffer is from 10 mM to 500 mM.

In some embodiments, the second agent comprises:
20 mM glycine buffer pH 7.4,
0.25% nano-microsphere (both first and second nano-microspheres), and
0.1% NaN₃;

the average particle size of the nano-microspheres is 450 nm.

According to another aspect of the present application, provided is a method for preparation of a nano-microsphere, comprising the steps of:
a first step, including:
   1.1) activating the first nano-microsphere to obtain an activated nano-microsphere;
   1.2) coupling the first antibody onto the activated nano-microsphere to obtain the first antibody-nano-microsphere conjugate;
   1.3) blocking the nano-microsphere resulting from step 1.2),
a second step, including:
   2.1) optionally, cross-linking the second antibody and the spacer arm molecule to obtain a complex of the second antibody and the spacer arm molecule;
   2.2) coupling the second antibody (or the complex resulting from step 2.1) onto the second nano-microsphere to obtain the second antibody-nano-microsphere conjugate;
   2.3) blocking the nano-microsphere resulting from step 2.2), and
a third step: mixing the nano-microspheres resulting from the first and second steps.

In other embodiments, the first and second steps are performed in parallel order or in an interchangeable order.

In some embodiments, the “activating” is performed with one or a combination of the reagent(s) selected from the group consisting of: 4-hydroxyethyl piperazine ethanesulfonic acid, sodium bicarbonate, sodium carbonate, ethyl dimethylamine propyl carbodiimide, hexamethylenediamine, 3,3′-diaminopropylimine and glutaraldehyde.

In some embodiments, in step 1.1): activating the first nano-microsphere with ethyl dimethylamine propyl carbodiimide to obtain an activated nano-microsphere. Preferably, ethyl dimethylamine propyl carbodiimide is dissolved in 20 mM pH 7.0 HEPES buffer at a concentration of 1 mg/ml. Preferably, the nano-microsphere is activated at 35 to 40° C. The concentration of the activated nano-microsphere is 5 mg/ml.

In a specific embodiment, in step 1.1): adding 5 mg/ml nano-microsphere dissolved in 20 mM pH 7.0 HEPES buffer into 0.1 mg/ml ethyl dimethylamine propyl carbodiimide, for activation at room temperature for 0.5 to 1 hour to obtain an activated nano-microsphere.

In some embodiments, in step 1.2): coupling the first antibody onto the activated nano-microsphere to obtain the first antibody-nano-microsphere conjugate. The first antibody is murine anti-human fPSA monoclonal antibody. In some embodiments, adding 0.1 mg/ml murine-anti-human fPSA monoclonal antibody dissolved in 20 mM pH 7.0 HEPES buffer into the activated nano-microsphere for reaction at 37° C. for 2 to 3 hours, such that the murine-anti-human fPSA monoclonal antibody is conjugated onto the activated nano-microsphere, resulting in the first antibody-nano-microsphere conjugate.

In some embodiments, in step 1.3): blocking the product of step 1.2) with a blocking solution comprising BSA and Tween 20 such that the part on nano-microsphere surface unbound with fPSA monoclonal antibody is blocked. Preferably, blocking the nano-microsphere resulting from step 1.2) with blocking solution comprising 1% BSA and 1% Tween 20 for 2 hours.

In some embodiments, after step 1.3), the resulting nano-microsphere is centrifuged and resuspended in the above buffer, preferably 20 mM pH 7.4 HEPES. Preferably, the concentration of the nano-microsphere is 0.25%, and optionally a preservative such as 0.1% NaN₃ can be added.

In some embodiments, in step 2.1), cross-linking the second antibody and glutaraldehyde (or an inert protein) to obtain a complex of the activated second antibody and glutaraldehyde (or an inert protein). In some specific embodiments, glutaraldehyde is dissolved in 20 mM pH 9.0 carbonic acid buffer at a concentration of 0.1 mg/ml. Preferably, the cross-linking is performed at 20 to 30° C. The concentration of the second antibody is 0.1 mg/ml. In a specific embodiment, in step 2.1), adding 0.1 mg/ml of the second antibody dissolved in 20 mM pH 9.0 carbonic acid buffer into 0.1 mg/ml glutaraldehyde for activation at 18-25° C. for 2-3 hours to obtain a complex of the second antibody and glutaraldehyde.

In some embodiments, in step 2.2), coupling the complex of the second antibody and glutaraldehyde onto the second nano-microsphere to obtain the second antibody-nano-microsphere conjugate. The second antibody is a single domain antibody, which specifically recognizes the complex formed by the first antibody and fPSA (instead of recognizing fPSA itself). In some embodiments, adding the complex of the second antibody and glutaraldehyde dissolved in 20 mM pH9.0 carbonic acid buffer into the second nano-microsphere for reaction at 18-25° C. for 2 to 3 hours so as to conjugate the second antibody onto the second nano-microsphere to obtain the second antibody-nano-microsphere conjugate.

In some embodiments, in step 2.3), blocking the product of step 2.2) with a blocking solution comprising BSA and Tween 20 such that the part on nano-microsphere surface unbound with fPSA monoclonal antibody is blocked. Preferably, blocking the nano-microsphere resulting from step 2.2) with blocking solution comprising 1% BSA and 1% Tween 20 for 2 hours.

In some embodiments, after step 2.3), the resulting nano-microsphere is centrifuged and resuspended in the above buffer, preferably 20 mM pH 7.4 HEPES. Preferably, the concentration of the nano-microsphere is 0.25%, and optionally a preservative such as 0.1% NaN₃ can be added.

In some embodiments, in the third step, the first antibody-nano-microsphere is mixed with the second antibody-nano-microsphere so that the ratio by weight of the two is from 1:4 to 1:1, for example, 1:4, 1:3.5, 1:3, 1:2.5, 1:2, 1:1.5, 1:1, and a range between any of the above. The mixture resulting from the third step is used as the second reagent in the detection kit of the present application.

In some embodiments, the nano-microsphere is a carboxyl- or amino-modified microsphere.

According to yet another aspect of the present application, there is provided a nano-microsphere bound to an fPSA-relating antibody, which is obtainable by the preparation method of the present application.

According to yet another aspect of the present application, there is provided a detection reagent, comprising the nano-microsphere as defined above.

In specific embodiments, there is provided a detection reagent comprising the first antibody-nano-microsphere and the second antibody-nano-microsphere as defined above.

According to yet another aspect of the present application, there is provided use of the first antibody-nano-microsphere conjugate and the second antibody-nano-microsphere conjugate as described above in the preparation of a reagent.

### DESCRIPTION OF THE DRAWINGS

FIG. 1: Comparison of standard curves between reagents prepared by the method of the present application and the reagents prepared by control method. ▪ The method of the present application; • control method 1; ♦ control method 2; ♦ control method 3.

FIG. 2: Correlation of serum measurements resulting from the kit of the present application and that from Chemiluminescence Immunoassay.

### DETAILED DESCRIPTION OF THE INVENTION

In order to make the present application easy to be understood, the present application will be further described with reference to specific embodiments below. “%” refers to w/v unless otherwise specified. The specific materials used in the embodiments of the present application and their sources are provided below. It should be understood, however, that these are exemplary only and are not intended to be limiting. Materials with the same or similar types, models, qualities, properties or functions as that of the following reagents and instruments can be used to implement the technical solutions of the present application.

### EXAMPLES

### Example 1: Preparation of fPSA Detection Kit

1. The first reagent comprising:

| | |
|---|---|
| HEPES | 50 mM |
| NaCl | 0.15M |
| NaN3 | 0.1% |
| BSA | 0.1% |
| Tween 20 | 0.1% |
| AEO7 | 0.3% |
| polyethylene glycol 6000 | 1%; |
| pH 7.3. | |

2. The second reagent was prepared as follows:

2.1 Preparation of the first antibody-nano-microsphere:
1) Ethyl dimethylamine propyl carbodiimide was dissolved with 20 mM HEPES buffer (pH 7.0) at 18-25° C., resulting in ethyl dimethylamine propyl carbodiimidea at a final concentration of 1 mg/ml;
2) 10 ml of 450 nm 10% (by weight) latex solution was diluted with 20 mM HEPES solution (pH 7.0) at 18-25° C. so that the latex concentration was 0.5% by weight;
3) 10 ml of 1 mg/ml EDAC solution dissolved in 20 mM HEPES solution (pH 7.0) was added, and stirred at 37° C. for 0.5 h to obtain activated nano-microspheres;
4) A murine anti-human fPSA monoclonal antibody was diluted with 10 ml of 20 mM pH 7.0 HEPES buffer to 0.1 mg/ml, added into the above activated nano-microspheres, and stirred at 37° C. for reaction for 2 h to obtain the first antibody-nano-microsphere conjugate;
5) 20 ml blocking solution (a solution comprising 1% BSA and 1% Tween 20) was added and stirred at 37° C. for reaction for 2 h;
6) The supernatant was discarded after centrifugation, 400 ml of 20 mM HEPES solution (pH 7.4) was added to obtain 0.25% latex, and 0.1% preservative was added and ultrasonically dispersed to obtain the first antibody-nano-microsphere.

2.2 Preparation of the second antibody-nano-microsphere:
1) Glutaraldehyde was dissolved with 20 mM carbonic acid buffer (pH 9.0) at 18-25° C. to make a final concentration of glutaraldehyde at 0.1 mg/ml;
2) 4 ml of the second antibody at a concentration of 5 mg/ml was diluted with 20 mM carbonic acid solution (pH 9.0) at 18-25° C. to make a final concentration of the second antibody at 0.1 mg/ml;
3) The second antibody solution resulting from step 2) was slowly dropwise added into the glutaraldehyde solution resulting from step 1), while stirring constantly at 18-25° C., and further stirred for another 3 h once the dropwise addition was completed;
4) The solution resulting from step 3) was placed in a dialysis bag with a molecular weight cut-off of 14,000, and dialysis was performed against 20 mM pH9.0 carbonic acid buffer to remove the uncrosslinked reactants to obtain a complex of the second antibody and glutaraldehyde;
5) 10 ml of 450 nm 10% (by weight) latex solution was diluted with 20 mM carbonic acid solution (pH 9.0) at 18-25° C. so that the latex concentration was 0.5% by weight;
6) The complex of the second antibody and glutaraldehyde was mixed well with the latex, reacted at 18-25° C. for 3 hours, and then 5 ml of blocking solution (a solution comprising 1% BSA and 1% Tween 20) was added for blocking for 2 hours;
7) The supernatant was discarded after centrifugation, the latex resulting from step 6) was diluted with 20 mM HEPES solution (pH 7.0) to 0.25%; 0.1% preservative was added, and ultrasonically dispersed to obtain the second antibody-nano-microsphere.

2.3 The prepared first and second antibody-nano-microspheres were mixed at a ratio of 1:1 by volume to obtain the second reagent.

3. Preparation of the reference calibrator:

3.1 The composition of the buffer for preparing the reference calibrator was as follows:

| | |
|---|---|
| HEPES | 50 mM |
| NaCl | 0.15M |
| NaN3 | 0.1% |
| anhydrous ethanol | 1% |
| BSA | 3%; |
| the rest was deionized water. | |

3.2 The pure fPSA was added into the above buffer, based on the concentrations required by the reference calibrators, to prepare fPSA reference calibrators with concentrations of 10 ng/ml, 20 ng/ml, 100 ng/ml, 500 ng/ml and 1000 ng/ml.

### Example 2: Control Preparation Method 1 (the Two Antibodies were Mixed and Coated on the Microsphere Together)

1. The preparation of the first reagent was the same as in Example 1.

2. The second reagent was prepared as follows:
1) Ethyl dimethylamine propyl carbodiimide was dissolved with 20 mM HEPES buffer (pH 7.0) at 18-25° C., resulting in ethyl dimethylamine propyl carbodiimidea at a final concentration of 1 mg/ml;
2) 10 ml of 450 nm 10% (by weight) latex solution was diluted with 20 mM HEPES solution (pH 7.0) at 18-25° C. so that the latex concentration was 0.5% by weight;
3) 10 ml of 1 mg/ml EDAC solution dissolved in 20 mM HEPES solution (pH 7.0) was added, and stirred at 37° C. for 0.5 h to obtain activated nano-microspheres;
4) The first and second antibodies were separately diluted with 5 ml 20 mM pH 7.0 HEPES buffer to 0.1 mg/ml and mixed well, added into the above activated nano-microsphere, and stirred at 37° C. for reaction for 2 h;
5) 20 ml blocking solution (a solution comprising 1% BSA and 1% Tween 20) was added and stirred at 37° C. for reaction for 2 h;
6) The supernatant was discarded after centrifugation, 400 ml of 20 mM HEPES solution (pH 7.4) was added to obtain 0.25% latex, and 0.1% preservative was added and ultrasonically dispersed to obtain the second reagent.

3. Preparation of the reference calibrator: the same as Example 1.

### Example 3: Control Preparation Method 2 (without the First Antibody)

1. The preparation of the first reagent was the same as in Example 1.

2. The second reagent was prepared as follows:
1) Glutaraldehyde was dissolved with 20 mM carbonic acid buffer (pH 9.0) at 18-25° C. to make the final concentration of glutaraldehyde at 0.1 mg/ml;
2) 2 ml of the second antibody at a concentration of 5 mg/ml was diluted with 20 mM carbonic acid solution (pH 9.0) at 18-25° C. to make the final concentration of the second antibody at 0.1 mg/ml, and mixed well;
3) The antibody solution of step 2) was slowly dropwise added into the glutaraldehyde solution resulting from step 1), while stirring constantly at 18-25° C., and further stirred for another 3 h after the dropwise addition was completed;
4) The solution resulting from step 3) was placed in a dialysis bag with a molecular weight cut-off of 14,000, and dialysis was performed against 20 mM pH9.0 carbonic acid buffer to remove the uncrosslinked reactants to obtain a complex of the antibody and glutaraldehyde;
5) 10 ml of 450 nm 10% (by weight) latex solution was diluted with 20 mM carbonic acid solution (pH 9.0) at 18-25° C. so that the latex concentration was 0.5% by weight;
6) The complex of the antibody and glutaraldehyde was mixed well with the latex, reacted at 18-25° C. for 3 hours, and then 5 ml of blocking solution (a solution comprising 1% BSA and 1% Tween 20) was added for blocking for 2 hours;
7) The supernatant was discarded after centrifugation, the latex resulting from step 6) was diluted with 20 mM HEPES solution (pH 7.0) to 0.25%, 0.1% preservative was added, and ultrasonically dispersed to obtain the second reagent.

3. Preparation of the reference calibrator: the same as Example 1.

### Example 4: Control Preparation Method 3 (the Coating Method for the First Antibody was Exchanged with that for the Second Antibody)

1. The preparation of the first reagent was the same as in Example 1.

2. The second reagent was prepared as follows:

2.1 Preparation of the first antibody-nano-microsphere:
1) Glutaraldehyde was dissolved with 20 mM carbonic acid buffer (pH 9.0) at 18-25° C. to make the final concentration of glutaraldehyde at 0.1 mg/ml;
2) 4 ml of the first antibody at a concentration of 5 mg/ml was diluted with 20 mM carbonic acid solution (pH 9.0) at 18-25° C. to make the final concentration of the first antibody at 0.1 mg/ml;
3) The first antibody solution resulting from step 2) was slowly dropwise added into the glutaraldehyde solution resulting from step 1), while stirring constantly at 18-25° C., and further stirred for another 3 h after the dropwise addition was completed;
4) The solution resulting from step 3) was placed in a dialysis bag with a molecular weight cut-off of 14,000, and dialysis was performed against 20 mM pH9.0 carbonic acid buffer to remove the uncrosslinked reactants to obtain a complex of the first antibody and glutaraldehyde;
5) 10 ml of 450 nm 10% (by weight) latex solution was diluted with 20 mM carbonic acid solution (pH 9.0) at 18-25° C. so that the latex concentration was 0.5% by weight;
6) The complex of the first antibody and glutaraldehyde was mixed well with the latex, reacted at 18-25° C. for 3 hours, and then 5 ml of blocking solution (a solution comprising 1% BSA and 1% Tween 20) was added for blocking for 2 hours;
7) The supernatant was discarded after centrifugation, the latex resulting from step 6) was diluted with 20 mM HEPES solution (pH 7.0) to 0.25%, 0.1% preservative was added, and ultrasonically dispersed to obtain the first antibody-nano-microsphere.

2.2 Preparation of the second antibody-nano-microsphere:
1) Ethyl dimethylamine propyl carbodiimide was dissolved with 20 mM HEPES buffer (pH 7.0) at 18-25° C., resulting in ethyl dimethylamine propyl carbodiimidea at a final concentration of 1 mg/ml;
2) 10 ml of 450 nm 10% (by weight) latex solution was diluted with 20 mM HEPES solution (pH 7.0) at 18-25° C. so that the latex concentration was is 0.5% by weight;
3) 10 ml of 1 mg/ml EDAC solution dissolved in 20 mM HEPES solution (pH 7.0) was added, and stirred at 37° C. for 0.5 h to obtain activated nano-microspheres;
4) The second antibody was diluted with 10 ml of 20 mM pH 7.0 HEPES buffer to 0.1 mg/ml, added into the above activated nano-microspheres, and stirred at 37° C. for reaction for 2 h to obtain the second antibody-nano-microsphere conjugate;
5) 20 ml blocking solution (a solution comprising 1% BSA and 1% Tween 20) was added and stirred at 37° C. for reaction for 2 h;
6) The supernatant was discarded after centrifugation, 400 ml of 20 mM HEPES solution (pH 7.4) was added to obtain 0.25% latex, and 0.1% preservative was added and ultrasonically dispersed to obtain the second antibody-nano-microsphere.

2.3 The prepared first and second antibody-nano-microspheres were mixed at a ratio of 1:1 by volume to obtain the second reagent.

3. Preparation of the reference calibrator: the same as Example 1.

### Example 5: Detecting Procedures of the fPSA Detection Kit

TABLE 1
Detecting procedures of the present application

| Substance added | Blank control | Calibrator/Sample |
|---|---|---|
| Deionized water | 15 μl | — |
| Reference | — | 15 μl |
| calibrator/sample | | |
| The first reagent | 160 μl | 160 μl |

Mixed well and reacted at 37° C. for 4 min

| | | |
|---|---|---|
| The second reagent | 80 μl | 80 μl |

Mixed well and reacted at 37° C. for 30 s, the absorbance A1 was
recorded at a wavelength of 800 nm, then reacted for another 5 min, the
absorbance A2 was recorded at a wavelength of 800 nm, ΔOD₇₀₀ =
A2 − A1.

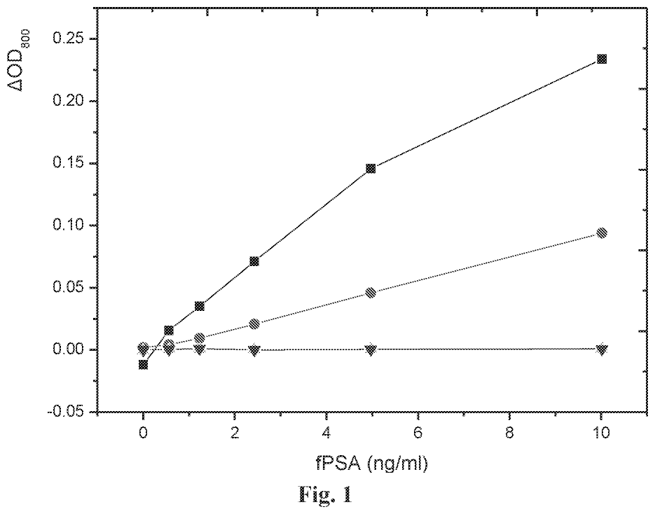

A standard curve was plotted by nonlinear fitting, such as spline, with the calibrator concentrations used as the horizontal axis, and the corresponding ΔOD800 used as vertical axis, as shown in FIG. 1.

By comparing the standard curves plotted with the reagents prepared by the control preparation method and those prepared by the present preparation method, it was found that the variation in the calibration absorbance values of the reagents of the present application is more significant, showing better sensitivity and linearity.

### Example 6: Linearity and Minimum Detection Limit of fPSA Detection Reagent

1. Test for Linearity:

A fPSA sample with high concentration was double-diluted by using methods known to those skilled in the art. Then, the diluted concentrations were measured by using the kit prepared by the method of the present application, the average value was calculated by measuring three times, and then was compared to the theoretical concentration so as to evaluate the linear deviation.

TABLE 2
Linear deviation

| | fPSA measurement value | | | | | |
|---|---|---|---|---|---|---|
| | (ng/ml) | | | | | |
| Fold of | the | the | the | | Theoretical | Deviation |
| Dilution | first | second | third | Average | value | % |
| 1 | 10.58 | 10.51 | 10.46 | 10.516 | 10.8582 | −3.1% |
| 0.8 | 8.76 | 8.75 | 8.76 | 8.756 | 8.7018 | 0.6% |
| 0.6 | 6.71 | 6.64 | 6.85 | 6.733 | 6.5454 | 2.9% |
| 0.4 | 4.58 | 4.59 | 4.57 | 4.58 | 4.389 | 4.4% |
| 0.2 | 2.33 | 2.34 | 2.33 | 2.333 | 2.2326 | 4.5% |
| 0.1 | 1.12 | 1.12 | 1.13 | 1.123 | 1.1544 | −2.7% |
| 0.08 | 0.9 | 0.88 | 0.9 | 0.893 | 0.93876 | −4.8% |
| 0.06 | 0.68 | 0.67 | 0.67 | 0.673 | 0.72312 | −6.88% |
| 0.04 | 0.46 | 0.43 | 0.46 | 0.45 | 0.50748 | −11.3% |
| 0.02 | 0.28 | 0.19 | 0.21 | 0.226 | 0.29184 | −22.3% |
| 0.01 | 0.13 | 0.14 | 0.16 | 0.14 | 0.1840 | −22.1% |
| 0 | 0.08 | 0.05 | 0.05 | 0.06 | 0.0762 | −21.3% |

It can be seen from Table 2 that the linear range of the kit of present application can reach 0.67-10 ng/ml. The linear range of the control kit according to Example 2 can reach 2-10 ng/ml, and the linearity for low-value samples is not as good as that of Example 1. Almost no reaction signal was detected by using the control kits according to Example 3 and Example 4, and these control kits cannot be used for linear detection.

The applicants unexpectedly found that in Example 4, no reaction signal could be detected, after merely exchanging the coating methods for the first and second antibody. Without being limited to a specific theory, it can be interpreted that when a complex is formed by the first antibody and the antigen, it is difficult for a larger antibody to recognize and get access to the epitope thereof due to steric hindrance. The applicants have unexpectedly noticed that when applying antibodies with smaller molecular weight (or antigen-binding fragments), (e.g. single domain antibodies) linked with a spacer arm molecule (glutaraldehyde, or inert carrier protein), the desired epitope can be reached and bound.

2. Minimum Detection Limit:

By using a method known to those skilled in the art, the blank solution and several low-concentration samples diluted with physiological saline were repeatedly measured for 15 times, and the changes in absorbance were recorded. Then the absorbance value of each sample was calculated after deducting the blank absorbance, and the mean and standard deviation were calculated. The minimum detection limit was calculated at a level of 99.7% probability. Three-fold of respective standard deviation for each sample was deducted from the mean of each sample, and then compared to 3-fold of the standard deviation for the blank solution. If the former was higher than the latter, we would determine that there was a 99.7% probability that the minimum absorbance was greater than the blank absorbance, thereby the method can quantitatively report the results. The measurement results are shown in Table 3.

TABLE 3
Test data for minimum detection limit

| | | Serum | Serum | Serum |
|---|---|---|---|---|
| ng/ml | Saline | sample 1 | sample 2 | sample 3 |
| 1 | −0.02 | 0.02 | 0.06 | 0.12 |
| 2 | 0.00 | 0.04 | 0.05 | 0.07 |
| 3 | 0.00 | 0.03 | 0.07 | 0.11 |
| 4 | 0.01 | 0.03 | 0.03 | 0.09 |
| 5 | −0.01 | 0.03 | 0.07 | 0.12 |
| 6 | −0.01 | 0.03 | 0.07 | 0.07 |
| 7 | 0.00 | 0.04 | 0.08 | 0.07 |
| 8 | 0.00 | 0.04 | 0.04 | 0.08 |
| 9 | −0.01 | 0.02 | 0.06 | 0.10 |
| 10 | 0.01 | 0.04 | 0.06 | 0.09 |
| 11 | 0.00 | 0.04 | 0.05 | 0.07 |
| 12 | 0.00 | 0.03 | 0.07 | 0.11 |
| 13 | 0.01 | 0.03 | 0.03 | 0.09 |
| 14 | −0.01 | 0.03 | 0.07 | 0.12 |
| 15 | −0.01 | 0.03 | 0.07 | 0.07 |
| Aver- | 0.00 | 0.03 | 0.06 | 0.09 |
| age | | | | |
| SD | 0.009486833 | 0.007888106 | 0.015238839 | 0.019888579 |
| cv % | | 24.65033243 | 25.97529421 | 21.61802013 |
| 3SD | | 0.023664319 | 0.045716518 | 0.059665736 |
| M + | 0.025793832 | | | |
| 3SD | | | | |
| M − | | 0.008335681 | 0.012950149 | 0.032334264 |
| 3SD | | | | |

It is known from Table 3 that when the sample concentration was 0.09 ng/ml, the result (deducting 3-fold of the standard deviation from the measured mean value) was higher than that of 3-fold of the standard deviation of saline, and the CV % was close to 20% in this case, hence 0.09 ng/ml would be deemed as the minimum detection limit of the detection reagents of present application.

The detection limit of the control kit of Example 2 was 1.5 ng/ml, and the detection limit was not as good as that of Example 1. Almost no reaction signal was detected using the control kits according to Example 3 and Example 4, and these control kits cannot be used for measuring detection limit.

### Example 7: Correlation of Measurements Resulting from the fPSA is Detection Reagents of the Present Application and from Chemiluminescence Immunoassay

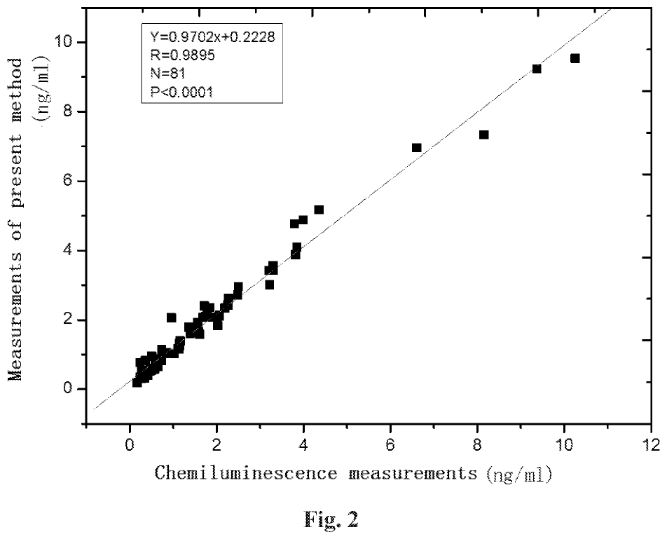

Serum samples were detected by using the fPSA detection kit of the present application and the chemiluminescence immunoassay in the prior art, respectively. The resulting measurements were compared (see FIG. 2), and regression analysis was performed to obtain the correlation R²=0.979, y=0.9702x+0.2228. It is shown that the present method has a good correlation with chemiluminescence immunoassay with respect to the measurement of serum fPSA.

The concept of the present application is that the second reagent comprises two antibodies: a first antibody and a second antibody. The first antibody can bind to fPSA in the sample to form the first antibody-antigen complex, while the second antibody does not bind to fPSA, but specifically binds to the first antibody-antigen complex. Therefore, the antibody located on the nano-microsphere reacts with fPSA in the sample to form a reticular complex, and the absorbance generated by the reaction is detected at 700 nm. The actual change in absorbance is proportional to the concentration of fPSA in the sample. The content of fPSA in the sample can be calculated quickly and effectively after plotting a calibration curve.

The main advantages of present application are:
(1) The first reagents comprises surfactant and polymerization accelerator, the optimized ratio of the two can increase the absorbance of the reaction while reducing the non-specific reaction.
(2) The second reagents comprises the first and second antibodies. The second antibody can specifically bind to the first antibody-antigen complex, and can directly react to generate a reticular complex, thereby generating an absorbance signal and improving the reaction signal, without using a competition method.
(3) By using the principle of latex-enhanced turbidimetric immunoassay, the reaction is homogeneous reaction within short reaction time, and results will be available within 10 minutes.
(4) Easy to operate; The requirements for instruments and equipment are not high, and not involving issues such as environmental protection and operator self-protection. At present, there is no commercially available kit for the determination of human serum or plasma fPSA concentration by latex-enhanced turbidimetric immunoassay. Compared to other detection methods, the present method is simple, fast, sensitive and reliable. It can be performed with common automatic or semi-automatic biochemical analyzers, and has a broad application and practical value.

The principle, main features and advantages of the present application have been shown and described above. The present application is not limited by the above embodiments. Without departing from the spirit and scope of the present application, there will be various modifications and improvements in the present application, and these modifications and improvements all fall within the claimed scope of the application.

1. A detection kit for free prostate-specific antigen, comprising:
a first reagent,
a second reagent; and
optionally, a quality control and/or calibrator;
wherein,
the first reagent comprises:
a surfactant, and
a buffer;
the second reagent comprises:
a first nano-microsphere coated with a first antibody,
a second nano-microsphere coated with a second antibody, and
a buffer;
the calibrator comprises human fPSA with known concentration;
the quality control comprises human fPSA with known concentration;
the first antibody is an anti-human fPSA antibody;
the second antibody is an anti-complex antibody, and the second antibody does not bind to human fPSA, but binds to the complex formed by the first antibody and human fPSA;
the buffer in the first reagent and in the second reagent is independently selected from the group consisting of: phosphate buffer, glycine buffer, HEPES buffer, IVIES buffer, boric acid buffer, acetate buffer, ammonium chloride buffer and the combination thereof;
the concentration of the buffer in the first reagent and in the second reagent is independently 10 mM to 500 mM;
the pH value of the buffer in the first reagent and in the second reagent is independently 6 to 8.

2. The detection kit for free prostate-specific antigen according to claim 1, wherein:
the first antibody is a monoclonal antibody or antigen-binding fragment thereof;
the second antibody is an antigen-binding fragment;
the monoclonal antibody is derived from: murine, leporidae, avian, caprinae, recombinant antibody;
the antigen-binding fragment is selected from the group consisting of: Fab, Fab′, F(ab′)2, scFv, Fv, dsFv and single domain antibody;
, the second antibody is linked to the second nano-microsphere via a spacer arm molecule;
the spacer arm molecule is glutaraldehyde or an inert carrier protein;
the inert carrier protein is selected from the group consisting of: serum albumin, thyroglobulin, ceruloplasmin, ovalbumin and polylysine.

3. The detection kit for free prostate-specific antigen according to claim 1, wherein the surfactant is selected from the group consisting of: fatty alcohol polyoxyethylene ether, Tween 20, Brij and the combination thereof;
the concentration of the surfactant is 0.01% to 3% w/v.

4. The detection kit for free prostate-specific antigen according to claim 1, wherein:
the nano-microsphere is formed by polymerization of one or more selected from the group consisting of: polystyrene, acrylic acid and acrylate;
the average particle size of the nano-microsphere is 400 nm to 500 nm, preferably d50 nm;
optionally, the first reagent further comprises one or more of the following:
a stabilizer of 0.05 to 0.2% w/v,
a preservative of 0.05 to 0.2% w/v,
a salt ion of 0.1 to 0.5M,
PEG of 0.5 to 2% w/v.

5. A method for preparation of a nano-microsphere, comprising the steps of:
a first step, including:
1.1) activating the first nano-microsphere to obtain an activated nano-microsphere;
1.2) coupling the first antibody onto the activated nano-microsphere to obtain the first antibody-nano-microsphere conjugate;
1.3) blocking the first antibody-nano-microsphere conjugate resulting from step 1.2), a second step, including:
2.1) optionally, cross-linking the second antibody and the spacer arm molecule to obtain a complex of the second antibody and the spacer arm molecule;
2.2) coupling the second antibody or the complex resulting from step 2.1) onto the second nano-microsphere to obtain the second antibody-nano-microsphere conjugate;
2.3) blocking the second antibody-nano-microsphere conjugate resulting from step 2.2),
a third step: mixing the first antibody-nano-microsphere conjugate with the second antibody-nano-microsphere conjugate,
wherein the first step and second step are performed in parallel or interchangeable;
the first antibody is an anti-human fPSA antibody;
the second antibody is an anti-complex antibody, and the second antibody does not bind to human fPSA, but binds to the complex formed by the first antibody and human fPSA.

6. The method for preparation of a nano-microsphere according to claim 5, wherein,
the nano-microsphere is formed by polymerization of one or more selected from the group consisting of: polystyrene, acrylic acid and acrylate;
the average particle size of the nano-microsphere is 400 nm to 500 nm;
the first antibody-nano-microsphere conjugate is mixed with the second antibody-nano-microsphere conjugate at a ratio of 1:4 to 1:1 by weight; or
the first antibody-nano-microsphere conjugate is mixed with the second antibody-nano-microsphere conjugate at a ratio of 1:4 to 1:1 by mass-of-substance; or
the first antibody-nano-microsphere conjugate is mixed with the second antibody-nano-microsphere conjugate at a ratio of 1:4 to 1:1 by concentration; or
the first antibody is a monoclonal antibody or antigen-binding fragment thereof;
the second antibody is an antigen-binding fragment;
the monoclonal antibody is derived from: murine, leporidae, avian, caprinae, recombinant antibody;
the antigen-binding fragment is selected from the group consisting of: Fab, Fab′, F(ab′)2, scFv, Fv, dsFv and single domain antibody;
the second antibody is linked to the second nano-microsphere via a spacer arm molecule;
the spacer arm molecule is glutaraldehyde or an inert carrier protein;
the inert carrier protein is selected from the group consisting of: serum albumin, thyroglobulin, ceruloplasmin, ovalbumin and polylysine.

7. The method for preparation of a nano-microsphere according to claim 5,
wherein the activation is performed with one or a combination of the reagent(s) selected from the group consisting of: 4-hydroxyethyl piperazine ethanesulfonic acid, sodium bicarbonate, sodium carbonate, ethyl dimethylamine propyl carbodiimide, hexamethylenediamine, 3,3′-diaminopropylimine and glutaraldehyde.

8. The method for preparation of a nano-microsphere according to claim 5, wherein,
the first step:
1.1) activating the first nano-microsphere with ethyl dimethylamine propyl carbodiimide to obtain an activated nano-microsphere;
1.2) adding a murine-anti-human fPSA monoclonal antibody into the activated nano-microsphere for reaction at 25-40° C. for 2 to 3 hours to obtain the first antibody-nano-microsphere conjugate;
1.3) blocking the first antibody-nano-microsphere conjugate resulting from step 1.2) with blocking solution,
the second step:
2.1) adding the second antibody dissolved in the buffer into glutaraldehyde for activation at 18-25° C. to obtain a complex of the second antibody and glutaraldehyde;
2.2) adding the complex of the second antibody and glutaraldehyde dissolved in the buffer into the second nano-microsphere for reaction at 18-25° C. for 2 to 3 hours to obtain the second antibody-nano-microsphere conjugate;
2.3) blocking the second antibody-nano-microsphere conjugate resulting from step 2.2) with blocking solution.

9. A nano-microsphere obtained by the method for preparation of a nano-microsphere according to claim 5.

10. A detection reagent, comprising the nano-microsphere of claim 9;
or, the detection reagent comprising:
a first nano-microsphere coated with a first antibody, and
a second nano-microsphere coated with a second antibody,
the first antibody is an anti-antigen antibody;
the second antibody is an anti-complex antibody;
the second antibody does not bind to the antigen, but binds to the complex formed by the first antibody and the antigen;
the first antibody is a monoclonal antibody or antigen-binding fragment thereof;
the second antibody is an antigen-binding fragment;
the monoclonal antibody is derived from: murine, leporidae, avian, caprinae, recombinant antibody;
the antigen-binding fragment is selected from the group consisting of: Fab, Fab′, F(ab′)2, scFv, Fv, dsFv and single domain antibody;
the second antibody is linked to the second nano-microsphere via a spacer arm molecule;
the spacer arm molecule is glutaraldehyde or an inert carrier protein;
and the inert carrier protein is selected from the group consisting of: serum albumin, thyroglobulin, ceruloplasmin, ovalbumin and polylysine.

11. The detection kit for free prostate-specific antigen according to claim 3, wherein the fatty alcohol polyoxyethylene ether is selected from the group consisting of: AEO7, AEO9, AEO3 and the combination thereof.

12. The detection kit for free prostate-specific antigen according to claim 1, wherein the calibrator comprises: 0 ng/ml, 0.5 ng/ml, 1 ng/ml, 2 ng/ml, 5 ng/ml or 10 ng/ml fPSA, a buffer, a stabilizer and a preservative.

13. The method for preparation of a nano-microsphere according to claim 6, wherein the first nano-microsphere is a carboxyl-modified nano-microsphere.

14. The method for preparation of a nano-microsphere according to claim 6, wherein the second nano-microsphere is an amino-modified nano-microsphere.

15. The method for preparation of a nano-microsphere according to claim 8, wherein in step 1.1) activating the first nano-microsphere with 1 mg/ml ethyl dimethylamine propyl carbodiimide dissolved in 20 mM pH 7.0 HEPES buffer at 35 to 40° C. to obtain an activated nano-microsphere, and the activated nano-microsphere has a concentration of 5 mg/ml.

16. The method for preparation of a nano-microsphere according to claim 8, wherein in step 1.2) adding 0.1 mg/ml murine-anti-human fPSA monoclonal antibody dissolved in 20 mM pH7.0 HEPES buffer into the activated nano-microsphere for reaction at 25-40° C. for 2 to 3 hours to obtain the first antibody-nano-microsphere conjugate.

17. The method for preparation of a nano-microsphere according to claim 8, wherein in step 1.3) blocking the first antibody-nano-microsphere conjugate resulting from step 1.2) with blocking solution comprising BSA and Tween 20 for 2 hours.

18. The method for preparation of a nano-microsphere according to claim 8, wherein in step 2.1) adding 0.1 mg/ml of the second antibody dissolved in 20 mM pH 9.0 carbonic acid buffer into 0.1 mg/ml glutaraldehyde for activation at 18-25° C. for 2-3 hours to obtain a complex of the second antibody and glutaraldehyde.

19. The method for preparation of a nano-microsphere according to claim 8, wherein in step 2.2) adding the complex of the second antibody and glutaraldehyde dissolved in 20 mM pH9.0 carbonic acid buffer into the second nano-microsphere for reaction at 18-25° C. for 2 to 3 hours to obtain the second antibody-nano-microsphere conjugate.

20. The method for preparation of a nano-microsphere according to claim 8, wherein in step 2.3) blocking the second antibody-nano-microsphere conjugate resulting from step 2.2) with blocking solution comprising BSA and Tween 20 for 2 hours.
